(19) United States
(12) Patent Application Publication
(10) Pub. No.: US 2016/0178630 A1
(43) Pub. Date: Jun. 23, 2016

(54) METHODS FOR PREDICTING OVERALL SURVIVAL OF CANCER PATIENTS BASED ON NUMBERS OF CIRCULATING TUMOR CELLS

(71) Applicant: Creatv MicroTech, Inc., Potomac, MD (US)

(72) Inventors: Daniel L. ADAMS, Basking Ridge, NJ (US); Cha-Mei TANG, Potomac, MD (US)

(73) Assignee: Creatv MicroTech, Inc., Potomac, MD (US)

(21) Appl. No.: 14/974,889

(22) Filed: Dec. 18, 2015

Related U.S. Application Data
(60) Provisional application No. 62/093,876, filed on Dec. 18, 2014.

(51) Int. Cl. G01N 33/574; G01N 15/10

(52) U.S. Cl. CPC G01N 33/57415; G01N 33/57492; G01N 15/10; G01N 2333/70589; G01N 2333/4742; G01N 2015/1062; G01N 2015/0065

(57) ABSTRACT

Cytokeratin (CK)+/CD45−/DAPI+ cells in the blood of cancer patients have been considered by most as circulating tumor cells (CTCs). Different methods of isolating CTCs results in a wide range of numbers and subgroups of CTCs from same patient. As a result, the clinical significance of the number of CTCs becomes cloudy. Provided herein is methodology to categorize CTCs into morphologically distinct subpopulations, and to use one of the subpopulations in methods for predicting overall survival of a patient having cancer.

### BACKGROUND

Many recent publications reporting the presence of hundreds, to thousands, of circulating tumor cells (CTCs) in the blood of cancer patients have raised questions regarding the prevalence of CTCs, as enumerated by the CellSearch® Test. Although CellSearch® detects clinically relevant CTCs, the ability to capture only EpCAM+CTCs has led to speculation that CellSearch® captures only limited subsets of CTCs. In contrast, alternative isolation approaches often capture large numbers of CTCs from similar patient blood samples and, not surprisingly, these alternative approaches have poor correlations to CellSearch®. Given these problems, the development of means for accurately determining the number of CTCs in a blood sample are needed. The present invention is directed to this and other important goals.

### BRIEF SUMMARY

The present invention is directed in a first embodiment to means and methods for isolating and identifying CTC subtypes in the blood of a subject. Particular means include CellSieve™ microfilters that have pore sizes which permit easy capture of CTCs from a sample. The CTCs isolated by CellSieve™ maintain good cell morphology and CellSieve™ microfilters have low fluorescence background. These features allow subtyping of the CTCs by morphology and staining. Three distinct CK+, histologically definable, staining patterns (filamentous, diffuse and punctate) have been identified using CellSieve™ microfilters. Additionally, the nuclear staining patterns of CK+CTCs isolated by CellSieve™ could be distinguished histologically as either apoptotic or pleomorphic.

The present invention is directed in a second embodiment to the use of one particular CTC subtype in the diagnosis of cancer. CTCs having a filamentous CK pattern and pleomorphic nuclear pattern had significant correlation with cells obtained using CellSearch®. These findings suggest that a subset of CTCs captured by CellSieve™ microfiltration is statistically correlated with CTCs obtained by CellSearch®. Thus, the prognostic implications of CTCs from CellSearch® may be applied to CellSieve™ microfiltration based capture systems.

The present invention is directed in a third embodiment to the use of one particular CTC subtype (PDCTCs) in predicting of overall survival in cancer patients over a 24 month period. In one aspect of this embodiment, the invention is directed to a method for predicting overall survival of a patient having cancer, comprising enumerating pathologically-defined circulating tumor cells (PDCTCs) in a blood sample from the patient, wherein when five or more PDCTCs are present in 7.5 ml of blood, the overall survival of the patient is predicted to be lower than a patient having cancer with four or less PDCTCs present in 7.5 ml of blood, and wherein the PDCTCs are cytokeratin (CK) 8, 18, 19⁺, CD45⁻, DAPI⁺ cells, possess a malignant nucleus, and display a filamentous CK pattern. In this aspect, the PDCTCs are enumerated by (i) isolating CTCs from the blood sample using a filter having pores of 7-8 microns in diameter and (ii) counting PDCTCs present in the isolated population of CTCs. In preferred aspects, the cancer is breast cancer.

### BRIEF DESCRIPTION OF THE DRAWINGS

FIG. 1. Flow chart of CTC isolation and identification using the CellSieve™ system.

FIGS. 2A-2X. Subpopulations of CTCs categorized based on cytological features of cytokeratin and DAPI on CellSieve™. (FIGS. 2A-2H) CTCs categorized as PDCTCs with (A, E) filamentous cytokeratin. (B, F) The nuclei are malignant, appearing with nuclear inclusions and margin irregularities. (C, G) Merged images. (D, H) Strong EpCAM+ expression. (FIGS. 2I-2P). CTCs categorized as EMTCTCs with (I, M) diffuse cytokeratin patterns. (J, N) Nuclei appears malignant with irregular nuclear contours but smooth margins and a regular oval shape. (K, O) Merged images. (L, P) Low/negative EpCAM expression. (FIGS. 2Q-2T) CTC categorized as EACTC with (Q) punctate cytokeratin. (R) Nucleus appears as malignant with an abnormal salt-and-pepper pattern. (S) Merged image. (T) EpCAM+ expression. (FIGS. 2U-2X) CTC categorized as LACTC with (U) punctate cytokeratin. (V) Nucleus also appears punctate, or blebbing. (W) Merged image. (X) Low/negative EpCAM expression.

FIG. 3. Examples of three different CTCs, shown as CellSearch Thumbnails from CellTracks Analyzer Ir.

FIGS. 4A-4B. Correlations of CK+ subpopulations identified by CellSieve™ filters versus enumeration by CellSearch®. (FIG. 4A) Linear regression curve plots between CK+ cells vs. CellSearch® showing a low correlation. (FIG. 4B) Linear regression curve plots between PDCTCs vs. CellSearch®, showing a high correlation.

FIGS. 5A-5B. (FIG. 5A) Examples of CK+/CD45− cells that could not be categorized into the 4 CTC subgroups and labeled as Atypical CK+ cells, and events not included in this study. Column A) Example of a CK+/CD45−/EpCAM+CAML Column B) Example of a DAPI+/CK+/EpCAM-event without visible cytoplasm, likely a “naked” nuclei of unknown origin. Column C) Example of a CK+/EpCAM+ event with no DAPI signal, likely a CTC with extruded nuclei, which are not included in this study. (FIG. 5B) Column D) Example of a CK+/CD45−/EpCAM+ cell which can be identified as a non-cancerous granulocyte which was not included in this study. Column E) Example of cell cluster >2 CTCs, but are counted as a single CTC for enumeration purposes, as is standard practice. Scale bars, 20 p.m.

FIG. 6. Percentage of EpCAM positivity in the CK+ populations.

FIGS. 7A-7C. Using the FDA clinical cut off (≧5 CTCs) to determine differences in clinical outcome between assays. (FIG. 7A) Using the FDA defined clinical cut-off of ≧5 CTCs versus ≧5 PDCTCs per 7.5 mL of blood we find disagreement for 6 patients (n=3 breast, n=3 prostate). (FIG. 7B) and (FIG. 7C) Kaplan-Meier curves for the overall survival of the patients that remained on study for two years (n=26). Of the 6 patients, with FDA defined disagreement between CellSearch® (B) and CellSieve™ (C), only 3 remained on study and were included in the plots.

FIGS. 8A-8F. Using the FDA clinical cut off (≧5 CTCs) to determine differences in clinical outcome between subcategories of CK+ cells as well as the PDCTC population with EpCAM. (FIG. 8A) Overall survival for the Total CK+ cell population. (FIG. 8B) Overall survival for the EMTCTC cell population. (FIG. 8C) Overall survival for the EACTC cell population. (FIG. 8D) Overall survival for the LACTC cell population. (FIG. 8E) Overall survival for the Atypical CK+ cell population. (FIG. 8F) Overall survival for the PDCTC cell population which was also EpCAM positive.

### DETAILED DESCRIPTION

A more complete appreciation of the present invention and many of the attendant advantages thereof will be readily obtained as the same becomes better understood by reference to the following detailed description when considered in connection with the accompanying drawings.

The matters defined in the description such as a detailed construction and elements are nothing but the ones provided to assist in a comprehensive understanding of the invention. Accordingly, those of ordinary skill in the art will recognize that various changes and modifications of the exemplary aspects described herein can be made without departing from the scope and spirit of the invention. Also, well-known functions or constructions are omitted for clarity and conciseness. Some exemplary aspects of the present invention are described below in the context of commercial applications. Such exemplary implementations are not intended to limit the scope of the present invention, which is defined in the appended claims.

As provided in the examples below, the present invention is based, in part, on a comparison study conducted to compare the results obtained using a microfiltration system (CellSieve™) with those of CellSearch® in the isolation and enumeration of circulating tumor cells (CTCs) captured from the blood of cancer patients. Like many non-EpCAM techniques, CellSieve™ isolated a greater number of Cytokeratin+(CK+)/CD45− cells than CellSearch®, and subsequent analysis showed a low correlation between the two systems. However, after sub-grouping cells based on distinct CK staining patterns and nuclear morphologies, a subpopulation was identified which is correlative to CellSearch®. Data is provided suggesting that although various morphologic CTCs with similar phenotypic expression patterns are present in the blood of cancer patients, clinically relevant cells isolated by CellSearch® can be isolated and identified using a non-EpCAM dependent approach.

Circulating Tumor Cells (CTCs)

Circulating tumor cells (CTCs) are cancer cells that originate from primary/metastatic solid tumors and found transiting the circulatory system. It has been postulated that CTCs represent a non-invasive method for treatment monitoring, subtyping, and tracking tumor progression in cancer patients. However, isolation of CTCs is challenging because of their extreme rarity, 1-10 CTCs among 10⁹ total blood cells, and compounded by the inherent heterogeneity of tumor cells. CTC isolation was first reported in 1869 and although great strides were made in increasing the efficiency of CTC isolation, a clinically validated prognostic assay was not developed until the advent of affinity based isolation. This clinical immunoassay, the CellSearch® CTC Test, captures CTCs from blood samples using ferrofluid nanoparticles conjugated with antibodies against the epithelial cell adhesion molecule (EpCAM). Often called the “standard” CTC Test, CellSearch® is the only FDA approved, clinically validated CTC assay proven to serve as an independent prognostic indicator of patient survival (OS) for breast, prostate and colorectal cancer patients.

CellSearch captures cells using a monoclonal antibody specific to EpCAM, and identifies CTCs using differential fluorescent antibodies to detect the presence of CK within a nucleus-containing intact cell, and the absence of CD45, as defining characteristics of CTCs. Though CellSearch® has the sensitivity to capture 1 CTC in 7.5 mL of blood, it only captures cells in <78% of metastatic carcinomas. As such, concerns have been raised as to whether the assay definition of CTCs is too restrictive and underestimates the number of true CTC events. To account for this underestimation, a number of techniques are being developed to increase capture efficiency by either altering the capture antibodies, or by forgoing affinity capture all together. To date, these techniques have failed to identify the CellSearch® CTC populations based on presence of CK, or EpCAM, and have shown neither correlation nor equivalency. Often, it is theorized that the inability to correlate these two techniques is a result of tumor cells losing their EpCAM expression, or cytokeratin expression, possibly through EMT processes.

Size exclusion, such as through the use of microporous filters, is a technique for isolating CTCs irrespective of their surface marker expression that has been shown to capture far greater numbers of CTCs than CellSearch®, at times, into the thousands per milliliter. This approach was first used over 50 years ago and was recently refined for greater clinical utility. However, commercial filters used for isolating CTCs can be quite imprecise and highly variable. Recent advances in microfabrication have allowed for the commercial production of precision microporous filters, which have overcome some of the previous issues, such as low porosity and high pressure. One such microfilter is the CellSieve™ microfilter, produced with precision pores arranged in arrayed patterns, giving the filters high porosity under low pressure. It has been shown that a low pressure filtration system can isolate circulating cells while preserving fine intracellular architecture, such as cytoskeletal structures, for in depth analysis using the CellSieve™ technology.

In an exploratory study, CK+/CD45− cells, with DAPI positivity (DAPI+), were isolated and enumerated from 30 breast and prostate cancer patients. Duplicate samples were run in parallel at different locations, using both CellSearch® and CellSieve™ platforms. It was found that CellSieve™ filters captured greater numbers of CK+/CD45−/DAPI+ cells than CellSearch®, findings that are consistent with other studies using size exclusion. After identifying CK+/CD45−/DAPI+ cells and EpCAM+CK+/CD45−/DAPI+ cells on CellSieve™, neither of which showed correlation to CellSearch®, and realizing that many previous studies focusing on EpCAM positivity in CTCs have failed to resolve the enumeration discrepancies versus CellSearch®, characterization of the distinct morphological features of the CK+/CD45−/DAPI+ cells was conducted. Starting with the cytology-based FDA definition of CTCs (e.g., positive fluorescent staining of CK 8, 18 and 19, CD45−, a diameter >4×4 μm, and a DAPI+ nucleus 50% of which is contained within the CK border), it was found the CTCs isolated by CellSieve™ express three distinct, histologically-definable, CK staining patterns, namely filamentous, diffuse and punctate. Additionally, the nuclear staining patterns of CTCs isolated by CellSieve™ could be distinguished histologically as either apoptotic or highly abnormal (e.g., high pleomorphism, non-uniform margins and unusually large size). Using these criteria, five distinct CK+/CD45−/DAPI+ subpopulations isolated by CellSieve™ were identified. Comparison analyses found that one main CK+/CD45−/DAPI+ subpopulation was highly correlative to the CellSearch® Test (R²=0.91, p=3.18*10⁻¹⁶), and this correlation was not dependent on EpCAM positivity.

These findings suggest that microfiltration of blood samples from cancer patients are indeed capturing a larger variety of CK+ expressing circulating cells (epithelial-like) than the CellSearch® system and furthermore, the clinically prognostic CTC population enumerated by CellSearch® may be characterized using a microfiltration approach followed by detailed cytometric analysis. Unlike previous studies on this subject, which have never found correlations to the CellSearch® subtype, an attempt was not made to determine the underlying functional biology of these CK+ expressing cells by comparing the expression of levels of various biomarkers. Described here is that characterization and categorization of CK+/CD45−/DAPI+ cells captured by microfiltration based on their CK and nuclear morphologic patterns numerically correlate to the prognostically valuable CellSearch® CTC subtype, which interestingly, does not seem dependent on EpCAM staining.

As indicated above, and discussed in the examples below, in one embodiment the invention is directed methods for predicting overall survival of a patient having cancer. The method is based on enumerating a specific CTC subpopulation in the blood of the subject and based on the number of these cells found in a given volume of blood, predicting the overall survival of that subject.

In particular, the invention is directed to a method for predicting overall survival of a patient having cancer, comprising enumerating pathologically-defined circulating tumor cells (PDCTCs) in a blood sample from the patient. For example, when five or more PDCTCs are present in 7.5 ml of blood of a subject having breast cancer, the overall survival of the patient is predicted to be lower than a patient having breast cancer with four or less PDCTCs present in 7.5 ml of blood. The number of PDCTCs that are used for the cut off will vary depending on the identity of the cancer.

It should be apparent to the skilled artisan that the noted method can be varied both in the volume of blood to be collected and screened, and in the number of PDCTCs that need to be in the sample in order to make the survival prediction. However, in the case of breast cancer, for example, the ratio of 5 cells per 7.5 mls of blood would be maintained.

In this method, the PDCTCs are defined as cells that are cytokeratin (CK) 8, 18, 19⁺, CD45⁻, and DAPI⁺ cells, possess a malignant nucleus, and display a filamentous CK pattern. In some instances, the malignant nucleus may also include nucleus in division.

In certain aspects, the CTCs are enumerated by (i) isolating CTCs from the blood sample and (ii) counting PDCTCs present in the isolated population of CTCs. Suitable means for isolating the CTCs from the blood include filters, microfluidic chips, red blood cell lysis, and white blood cell depletion methods. Any methods that can separate CTCs without damaging the cell morphology may be used. In preferred aspects, the CTCs are enumerated by (i) isolating CTCs from the blood sample using a filter having pores of 7-8 microns in diameter and (ii) counting PDCTCs present in the isolated population of CTCs. For the filter, the CellSieve™ microfilter produced by Creatv MicroTech is exemplary. CellSieve™ microfilters have pores 7-8 microns in diameter.

The PDCTCs in the isolated population of CTCs may be counted by first distinguishing the PDCTCs from other CTC subpopulations. As detailed in the examples herein, PDCTCs can be identified and distinguished using different combinations of antibodies and stains to reflect structural and morphologic characteristics of the cells. For example, isolated cells can be stained with an antibody cocktail consisting of FITC-anti-Cytokeratin 8, 18, 19; Phycoerythrin (PE) conjugated EpCAM; and Cy5-anti-CD45 to reveal cytokeratin (CK) 8, 18, 19⁺, CD45⁻ cells in the population. Fluoromount-G/DAPI can be used to reveal DAPI⁺ cells. A fluorescent microscope can be used to image the cells. For some assays a different marker can be used instead of EpCAM. Different fluorescent dyes can be used for the markers.

In preferred aspects, the cancer is breast cancer. The cancer includes, but is not limited to, breast cancer.

Other means for isolating and subtyping PDCTCs may be used that do not damage the cell morphology/structure. If the efficiencies of other CTC isolating and subtyping methods are close to 100% similar to the CellSieve™ microfiltration method, then the criteria of ≧5 PDCTCs correlating for short overall survival will apply to those other CTC isolation and subtyping methods as well.

### Materials and Methods

### Blood Sample Collection

In total, 30 patient peripheral blood samples from breast (n=21) and prostate (n=9) anonymized cancer patients were supplied through a collaborative agreement with Fox Chase Cancer Center (FCCC) and University of Maryland Baltimore (UMB), with written informed consent and according to the local IRB approval at each institution. In addition, 30 non-blinded healthy volunteer blood samples were collected in CellSave preservative tubes, with written informed consent and IRB approval by Western Institutional Review Board. Anonymized blood samples were drawn in tandem into two CellSave Tubes™ (˜9 mL). Within 72 hours, one tube (7.5 mL) was used to enumerate CTCs using CellSearch® at FCCC. The second tube (7.5 mL) was used to enumerate CTCs using CellSieve™ microfiltration at UMB or Creatv MicroTech. Results and patient identification from institutions were not shared or communicated until completion of the study.

### CellSieve™ Microfilter CTC Enumeration.

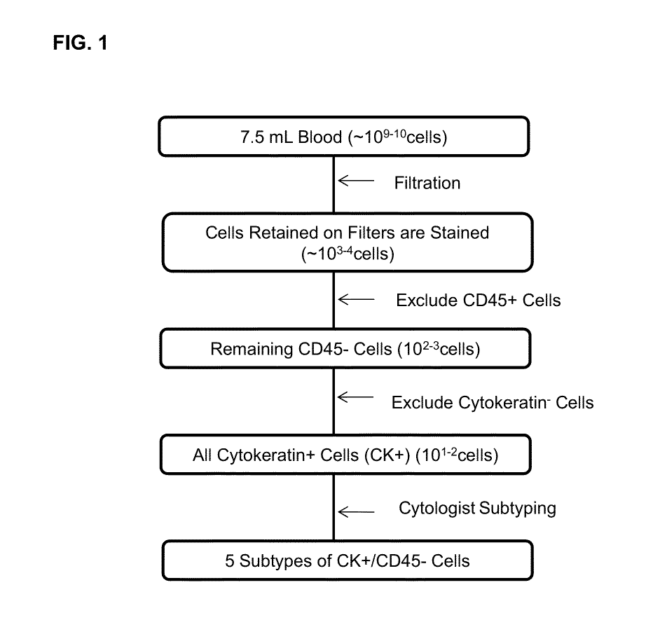

Each CellSieve™ Microfiltration Assay isolates CTCs based on size exclusion and identifies CTCs based on the histological cell architecture of cytokeratin, and nuclear morphologies. An overview of the process is shown in FIG. 1. 7.5 mL peripheral blood containing ˜10⁹⁻¹⁰ cells is filtered. ˜10³⁻⁴ cells are retained on the filters and are stained with DAPI, anti-CK and anti-CD45 antibodies. Stained cells on the filter are scanned for CD45 signal. ˜10²⁻³ of the cells are CD45−. Remaining cells are then scanned for CK+. ˜10-100 of the cells are CD45− and CK+. CK+/CD45− cells are imaged and subtyped by a trained cytologist into 5 distinct subpopulations based on cytokeratin and DAPI staining patterns.

The assay and reagents consist of CellSieve™ microfilter (≧160,000 pores in uniform array with 7 μm pore diameter within a 9 mm area), Prefixation buffer, a Postfixation buffer, a Permeabilization buffer, and an antibody cocktail. The low-pressure system used a filter holder assembly attached to a syringe pump drawn at 5 mL/min (as reported in WO 13/078409) or to a vacuum pump (Adams D L, et al. The systematic study of circulating tumor cell isolation using lithographic microfilters, RSC Adv. 2014, 4:4334-4342). Peripheral blood (7.5 mL) was collected in a CellSave tube, and diluted in a prefixation buffer before drawn through the filter. The filter was washed, postfixed and permeabilized. The captured cells were stained with an antibody cocktail consisting of FITC-anti-Cytokeratin 8, 18, 19; Phycoerythrin (PE) conjugated EpCAM; and Cy5-anti-CD45(5). Filters were then washed, placed onto a microscope slide and cover-slipped with Fluoromount-G/DAPI (Southern Biotech). An Olympus BX54WI Fluorescent microscope with Carl Zeiss AxioCam was used to image cells. Exposures were preset as 5 sec (Cy5), 2 sec (PE), 100-750 msec (FITC), and 10-50 msec (DAPI) for equal signal comparisons between cells. A Zen2011 Blue (Carl Zeiss) was used to process the images.

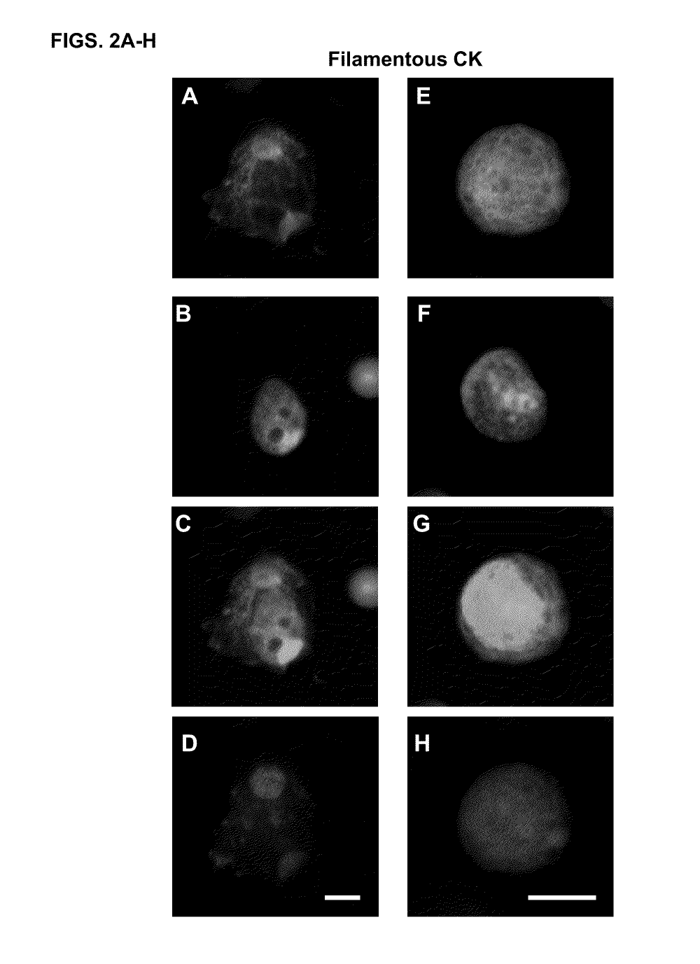

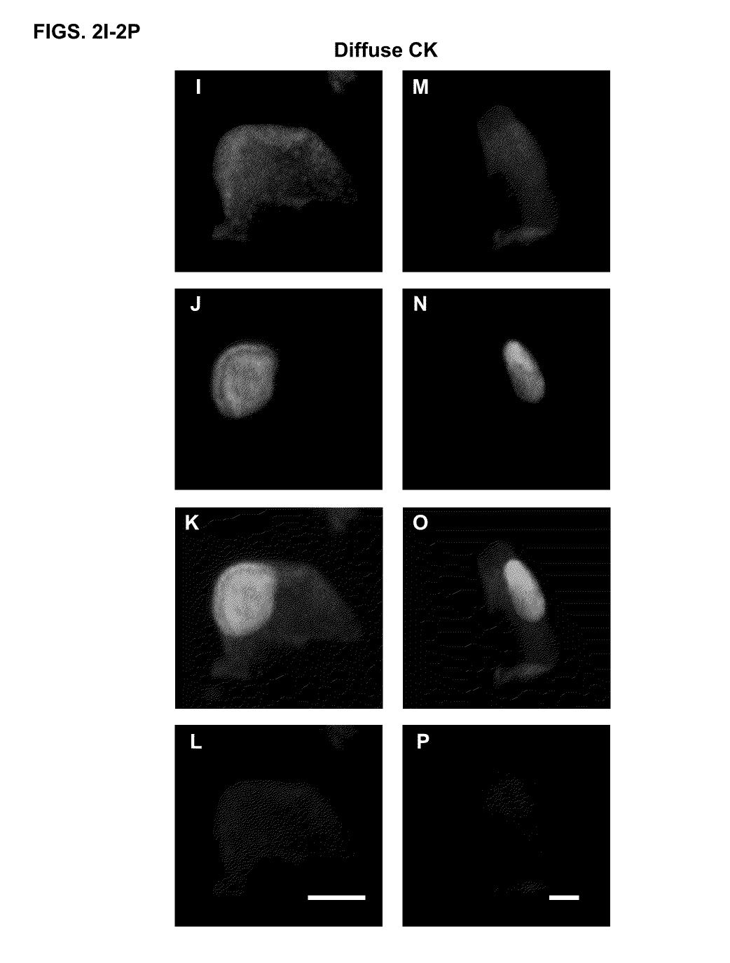

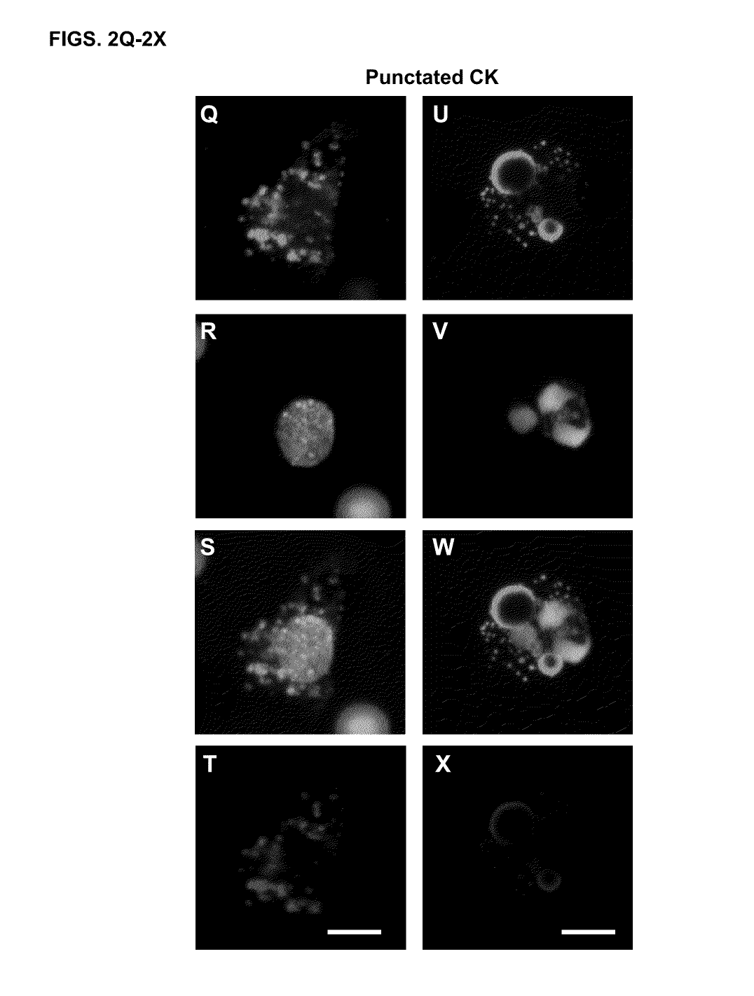

FIGS. 2A-X shows different ways CTCs are categorized based on the staining patterns of CK: filamentous, diffused, or punctuated cytokeratin. Scale bars are 10 μm.

CellSearch® CTC enumeration.

The CellSearch system was run following the Janssen protocols at FCCC. Immunomagnetic enrichment of CTCs using the CellTracks™ AutoPrep System. Peripheral blood samples collected in CellSave Preservative Tubes™ were maintained at ambient temperature. CellSearch™ Epithelial Cell kits (Janssen Diagnostics) were used for the isolation of CTCs. Isolations were performed on the CellTracks AutoPrep® System (Janssen Diagnostics). Data was collected and analyzed on the CellTracks Analyzer II® (Janssen Diagnostics).

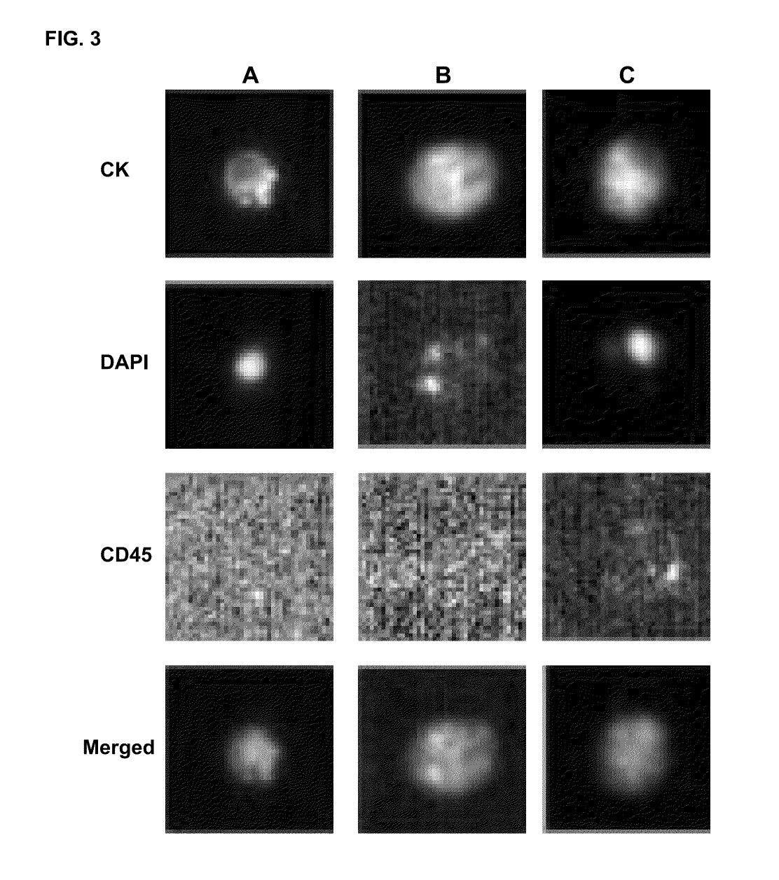

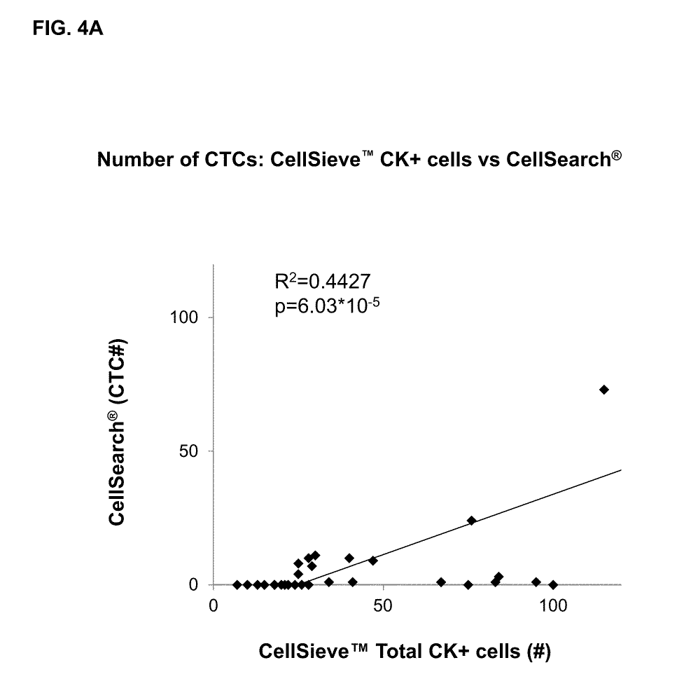

Briefly, anti-pan cytokeratin (CK 8, 18, 19)-PE, anti-CD45-APC and DAPI (CellSearch® Epithelial Cell kit reagents) were used to differentially label the CTC enriched product. Ferrofluid nanoparticles conjugated with anti-EpCAM antibodies captured CTCs from 7.5 mL of blood and were magnetically separated. Cells were washed, permeabilized, labeled with fluorescent antibodies, resuspended in Cell Fixative then loaded into a cartridge held in a magnetic holder (MagNest) which aligns the ferrofluid-captured cells. The Magnest was placed into a CellTracks Analyzer II® and the fluorescently labeled cells were imaged. Images were sorted using computer-assisted software selecting and presenting CK+ and DAPI+ events. A technician selected cells meeting the FDA criteria for CTCs, e.g. 1) expressing CK, 2) lacking CD45 and 3) containing a DAPI+ nucleus 50% which is contained within an intact CK+ perimeter. Examples of three different cells are shown in columns A-C of FIG. 3. Because the ferrofluid remains on the CK+ events, detailed cellular cytology was not possible and therefore solely presence, or absence, of fluorescence was used to identify CTCs. Scale bars are not given on the images from the CellTracks Analyzer II®.

### Statistical Methods

Linear regression plots were made using the enumerated counts from all subtypes of CK+/CD45− cells identified using CellSieve™ and the CTCs enumerated by CellSearch®. Spearman correlation coefficients were calculated for each CK+/CD45− subtype using MATLAB R2013A. Power analysis for sample size was calculated using previously published CVs using MATLAB R2013A.

Data analyses and correlations of CTC subtypes identified by CellSieve™ filters versus enumeration of CTCs from CellSearch® for EpCAM+, breast, prostate and combined patient samples are presented in Tables 1A-D.

TABLE 1A
The correlation table with associated slopes for various subtypes of CTCs, including a column for EpCAM
positive PDCTCs and a column combining the PDCTC and the EACTC groups (n = 30).

| | Total CK+ | | | | | Atypical CK+ | PDCTC+ | PDCTC+ |
|---|---|---|---|---|---|---|---|---|
| Breast plus | cells | PDCTC | EMTCTC | EACTC | LACTC | cells | EpCAM | EACTC |
| Prostate | vs. | vs. | vs. | vs. | vs. | vs. | vs. | vs. |
| Comparison | CellSearch ® | CellSearch ® | CellSearch ® | CellSearch ® | CellSearch ® | CellSearch ® | CellSearch ® | CellSearch ® |
| R² | 0.4427 | 0.9107 | 0.0108 | 0.6033 | 0.1311 | 0.0002 | 0.94 | 0.98 |
| p-value | 6.03 * 10⁻⁵ | 3.18 * 10⁻¹⁶ | 0.58 | 4.50 * 10⁻⁷ | 0.05 | 0.95 | 3.03 * 10⁻¹⁸ | 6.88 * 10⁻²⁵ |
| Slope of | 0.45x | 1.34x | −0.26x | 2.51x | 1.40x | 0.02x | 1.64x | 1.07x |
| curve | | | | | | | | |

TABLE 1B
The correlation between the CTC subcategories identified by the CellSieve ™ assay compared to the enumeration
of CTCs by the CellSearch ® assay for breast cancer patients and associated slopes (n = 21).

| | Total CK+ | | | | | Atypical CK+ | PDCTC+ | PDCTC+ |
|---|---|---|---|---|---|---|---|---|
| | cells | PDCTC | EMTCTC | EACTC | LACTC | cells | EpCAM | EACTC |
| Breast | vs. | vs. | vs. | vs. | vs. | vs. | vs. | vs. |
| Comparison | CellSearch ® | CellSearch ® | CellSearch ® | CellSearch ® | CellSearch ® | CellSearch ® | CellSearch ® | CellSearch ® |
| R² | 0.3812 | 0.9715 | 0.0076 | 0.6988 | 0.0049 | 0.0002 | 0.96 | 0.98 |
| p-value | 2.86 * 10⁻³ | 3.84 * 10⁻¹⁶ | 0.71 | 2.38 * 10⁶ | 0.76 | 0.95 | 3.06 * 10⁻¹⁴ | 1.48 * 10⁻⁶ |
| Slope of | 0.41x | 1.26x | −0.19x | 4.08x | 0.32x | 0.02x | 1.55x | 1.05x |
| curve | | | | | | | | |

TABLE 1C
The correlation between the CTC subcategories identified by the CellSieve ™ assay compared to the enumeration
of CTCs by the CellSearch ® assay for prostate cancer patients and associated slopes (n = 9).

| | Total CK+ | | | | | Atypical CK+ | PDCTC+ | PDCTC+ |
|---|---|---|---|---|---|---|---|---|
| | cells | PDCTC | EMTCTC | EACTC | LACTC | cells | EpCAM | EACTC |
| Prostate | vs. | vs. | vs. | vs. | vs. | vs. | vs. | vs. |
| Comparison | CellSearch ® | CellSearch ® | CellSearch ® | CellSearch ® | CellSearch ® | CellSearch ® | CellSearch ® | CellSearch ® |
| R² | 0.7308 | 0.9508 | 0.0559 | 0.7704 | 0.8015 | 0.0023 | 0.97 | 0.97 |
| p-value | 3.31 * 10⁻³ | 7.85 * 10⁻⁶ | 0.54 | 1.86 * 10⁻³ | 1.10 * 10⁻³ | 0.90 | 1.41 * 10⁻⁶ | 1.19 * 10⁻⁶ |
| Slope of | 0.61x | 2.06x | −3.90x | 1.88x | 2.60x | 0.07x | 2.07x | 1.12x |
| curve | | | | | | | | |

TABLE 1D
The correlations between the categories of CK+ cells with EpCAM positive expression compared with
CellSearch ®. Interestingly, using EpCAM phenotype expression gave no added statistical benefit. However we find that optimal
method equivalency was accomplished by adding both PDCTC and EACTC into one group.

| | Total CK+/EpCAM+ | | | | | Atypical CK+ |
|---|---|---|---|---|---|---|
| | cells | PDCTC/EpCAM+ | EMTCTC/EpCAM+ | EACTC/EpCAM+ | LACTC/EpCAM+ | cells/EpCAM+ |
| EpCAM+ | vs. | vs. | vs. | vs. | vs. | vs. |
| Comparison | CellSearch ® | CellSearch ® | CellSearch ® | CellSearch ® | CellSearch ® | CellSearch ® |
| R² | 0.6272 | 0.94 | 0.0051 | 0.2533 | 0.1304 | 0.0003 |
| p-value | 1.85 * 10⁻⁷ | 3.03 * 10⁻¹⁸ | 0.71 | 4.0 * 10⁻³ | 0.05 | 0.93 |
| Slope of curve | 0.86x | 1.64x | −0.53x | 2.5x | 2.3x | 0.04x |

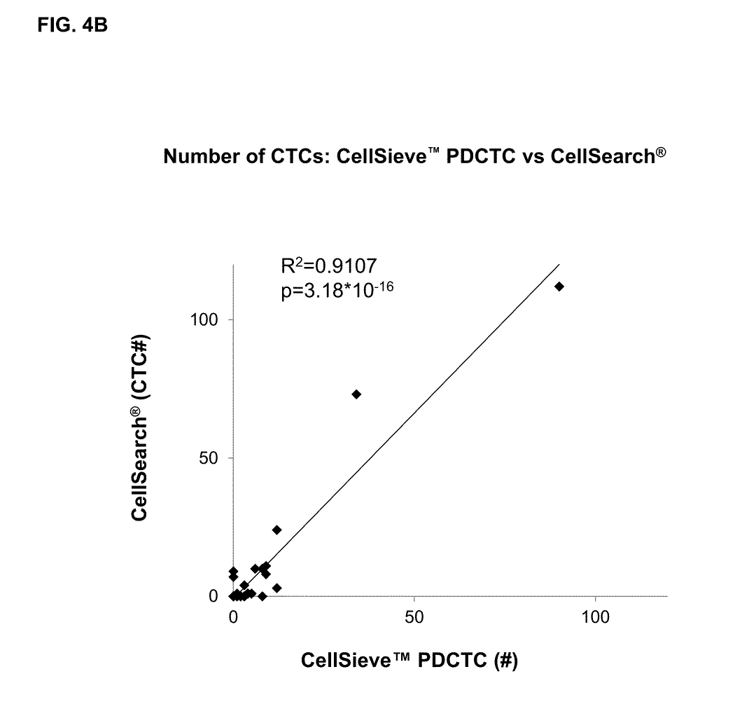

FIG. 4A is a linear regression curve plot between total CK+ cells vs. CellSearch® showing a low correlation. From Table 1, R²=0.4427, p-value=6.0×10⁻⁵, and slope of curve=0.45×. FIG. 4B is a linear regression curve plot between PDCTCs vs. CellSearch®, showing a high correlation. From Table 1, R²=0.9107, p-value=3.8×10⁻¹⁶, and slope of curve=1.34×.

### Results and Discussion

Since the CellSearch system utilizes a highly specific EpCAM-based approach to capture CTCs, it has been argued that it is insensitive to circulating epithelial cells which do not express EpCAM on their cell surface. Therefore, it is concluded that this technique has limited utility on broader patient cohorts and failings in capturing and identifying cancer stem cells which have undergone EMT, a heterogeneous process with no standardized definition. Alternative techniques, such as size based isolation, whole blood cell smears, electrophoresis, etc., attempt to increase sensitivity of CTC capture, typically while sacrificing specificity. Not surprisingly, less stringent techniques have been shown to capture far greater numbers of CK+ and EpCAM+ expressing cells from the blood of cancer patient samples, at times numbering thousands of CK+, or EpCAM+ expressing cells per milliliter. The greater number of CK+ expressing cells captured by these techniques is argued to be a result of greater efficiency of their approaches. However, the same clinically validated data provided by CellSearch® has yet to be reproduced by these alternative approaches and attempts to account for these discrepancies by evaluating the functional biology of the CK+ cell types by using additional biomarker information, such as EpCAM presence, have not yet yielded improved correlations with CellSearch®.

In an effort to reconcile the discrepancies between CK+ expressing cells captured using filtration techniques, and the prognostically significant enriched CK+ expressing cells identified as CTCs via CellSearch®, a detailed examination of all CK+ expressing cells captured by the CellSieve™ microfiltration system was performed. To directly compare the two techniques, only staining patterns of the standard CellSearch® detection markers were examined, including intact cells with cytokeratin, CD45, EpCAM, and nuclear DAPI, and not by adding additional marker systems nor including CK+ particles.

Cytokeratins are intermediate filament proteins expressed by epithelial derived cells and are prevalent in transformed epithelial cells, such as CTCs. These structures are extremely fine (˜10 nm diameter) and their morphologies can give information regarding apoptosis, structural integrity, and anaplasia. Since the CellSieve™ system has been shown to preserve internal cellular structures, detailed analysis of the distinct CK+ filament architecture can be performed.

The distinct CK+ staining pattern of cells captured by CellSieve™ can be readily identified as filamentous, diffuse and punctate and form the basis of CTC sub classification used in this study (FIG. 2). Filamentous CK is the classical and established example of epithelial intermediate filaments, with fibril like structures traversing though the interior of a cell (FIG. 2A, E). Diffuse CK is defined by a weak CK staining without observable filamentous patterns, usually this pattern is associated with epithelial-mesenchymal transition, though no universal definition of EMT transition currently exists (FIGS. 2I, M). Punctate CK staining can be attributed to the collapse of the cytoskeletal structure, in the early stages of apoptosis, which results in retraction of the cytokeratin filaments, referred to as blebbing (FIGS. 2Q, U). Cytokeratin blebbing has also been described in the CellSearch® Test Analyzer and typically counted as a CTC, though disagreements in the definitions between intact “granular” CTCs and cell particles do exist.

Nuclear morphology is another criteria used in identifying, grading and classifying cancer cells in both cancer biopsies and on the CellSearch® system. After filtration, abnormal nuclear patterns were identified that are typically seen in tumor cells (e.g., pleomorphism, non-uniform margins, unusually large size) (FIGS. 2B, F, J, N, R and V). These nuclear variations are a prerequisite for morphologically classifying CTCs and, in cases of punctate CK patterns the presence of these variations were used to identify cells undergoing early apoptotic or late apoptotic events. In early apoptotic CTCs, the CK+ staining is punctate; however, the nucleus is intact (FIGS. 2Q, R). In late apoptotic CTCs, the CK staining is punctate and the progressive apoptotic process has broken the nucleus apart, also called nuclear blebbing (FIGS. 2U, V). In either case, a DAPI positive signal within a CK+ signal is defined as a CTC on the CellSearch® Test Analyzer.

Based on the three CK+ staining patterns (filamentous, diffuse and punctate) and two nuclear staining pattern (malignant and punctate), four distinct subpopulations were identified which make up the total CK+/CD45− expressing cells classified as CTCs isolated by CellSieve™ and they are described in detail below as pathologically definable CTCs (PDCTCs), Epithelial-Mesenchymal Transition-like CTCs (EMTCTCs), early apopototic CTCs (EACTCs) and late apoptotic CTCs (LACTCs).
Pathologically definable CTCs (PDCTCs): 1) have strong filamentous CK+ signal, 2) have a DAPI+ nuclei with malignant pathologies. FIGS. 2A-H shows CTCs with filamentous cytokeratin, categorized as PDCTCs. FIGS. 2A and 2E show filamentous cytokeratin. FIGS. 2B and 2F show malignant nuclei, appearing with nuclear inclusions and margin irregularities. FIGS. 2C and 2G are merged images of cytokeratin and nuclei. FIGS. 2D and 2H show strong EpCAM+ expression.
Epithelial-Mesenchymal Transition-like CTCs (EMTCTCs): 1) have diffuse/non-filamentous and weak CK+ signal, 2) have a DAPI+ nuclei with malignant pathologies. FIGS. 2I-2H show CTCs with diffused cytokeratin, categorized as EMTCTCs. FIGS. 21 and 2M show diffuse cytokeratin patterns. FIGS. 2J and 2N show nuclei that appear malignant with irregular nuclear contours but smooth margins and a regular oval shape. FIGS. 2K and 20 are merged images of cytokeratin and nuclei. FIGS. 2L and 2P are low/negative EpCAM expression.
Early Apoptotic CTCs (EACTCs): 1) have a punctate CK+ signal, 2) have intact DAPI+ nuclei with malignant pathologies FIGS. 2Q-T shows a CTC with punctuated cytokeratin, categorized as EACTC. FIG. 2Q shows punctate cytokeratin. FIG. 2R shows a nucleus appearing as malignant with an abnormal salt-and-pepper pattern. FIG. 2S is a merged image of cytokeratin and the nucleus. FIG. 2T shows EpCAM+ expression.
Late Apoptotic CTCs (LACTCs): 1) have a punctate CK+ signal, 2) have a punctate nuclear DAPI+ staining FIGS. 2U-X show a CTC with punctuated cytokeratin, categorized as LACTC. FIG. 2U shows punctate cytokeratin. FIG. 2V show the nucleus appearing punctate, or blebbing. FIG. 2W is the merged images of cytokeratin and nucleus. FIG. 2X is the low/negative EpCAM expression.

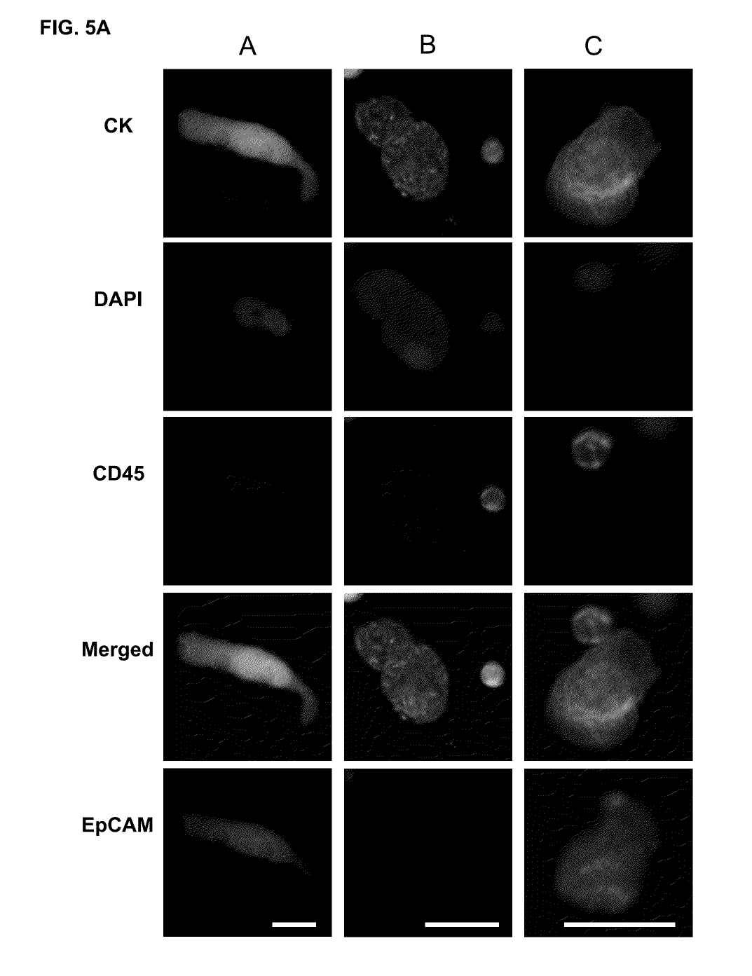

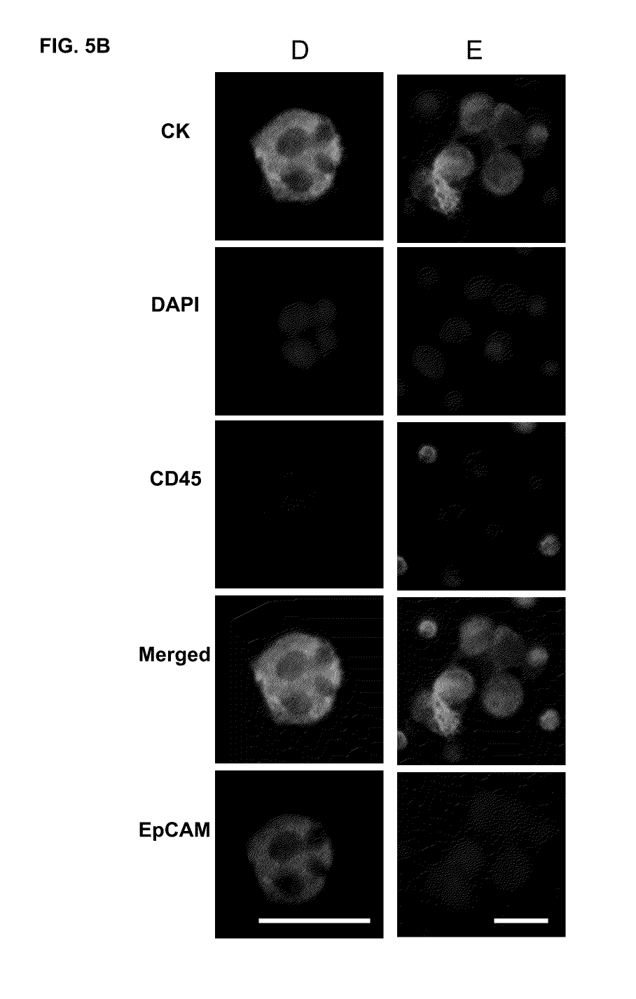

The CK+/CD45− cells in the four subpopulations ranged from high EpCAM positivity to low/negative positivity (FIGS. 2D, H, L, P, T and X), but were not a driving factor in concordance (Tables 1A-D). CK+/CD45− cells that could not be categorized into these four subpopulations were classified as “Atypical CK+ cells” and not counted as CTCs for this study (FIGS. 5A-D). These cells included CK+/CD45− cancer associated macrophage-like cells (CAMLs) (FIG. 5A) and DAPI+ and CK+ cells without visible cytoplasm (FIG. 5B). Other CK+/CD45− events not included in this study, as they do not meet the criteria of a CTC, include 1) CK+/CD45− events with no DAPI signal (FIG. 5C) and 2) CK+/CD45− cells which were identified as non-cancerous (e.g. granulocytes, macrophages, etc.) by a pathologist (FIG. 5D). Additionally, cell clusters/microemboli of ≧2 were counted as one CTC (FIG. 5E), following equivalence to CellSearch® enumeration.

In Table 2, the four CK+/CD45−CTC subpopulations, Atypical CK+ cells and the total CK+/CD45− cells are shown in comparison to CellSearch® enumeration, for the 30 duplicate patient samples. CellSieve™ captured 979 CK+/CD45− cells from 21 breast cancer patient blood samples compared to 162 CTCs captured by CellSearch®. Additionally, CellSieve™ captured 379 CK+/CD45− cells from nine prostate cancer patients, compared to 114 by CellSearch®. No CTCs, from the 30 healthy volunteer blood samples, were found on the CellSieve™ system. These data support previous publications regarding greater CTC capture from patient blood samples using size exclusion.

TABLE 2
CTCs enumerated by CellSearch ® and CK+ subpopulations identified by
CellSieve ™. CTCs isolated from duplicate samples of blood from prostate (PC) and breast (BC)
cancer patients. The columns from left to right show patient number, Classification of Malignant
Tumors (TMN) and the number of CTCs identified by CellSearch ®. The next six columns show
the number of CTC subpopulations, and the total number of CK+ cells identified by CellSieve ™.

| | | | | | | | Atypical | Total CK+ |
|---|---|---|---|---|---|---|---|---|
| | | | PDCTC | EACTC | LACTC | EMTCTC | CK+ cells | cells |
| Patient | TNM | CellSearch ® | CellSieve ™ | CellSieve ™ | CellSieve ™ | CellSieve ™ | CellSieve ™ | CellSieve ™ |
| BC1 | T2/N1/M1 | 0 | 0 | 0 | 0 | 45 | 55 | 100 |
| BC2 | TX/NX/MX | 0 | 0 | 0 | 5 | 0 | 10 | 15 |
| BC3 | TX/NX/M1 | 0 | 2 | 0 | 2 | 0 | 14 | 18 |
| BC4 | T2/N1/M0 | 0 | 2 | 0 | 2 | 2 | 7 | 13 |
| BC5 | TX/N2/M0 | 0 | 1 | 0 | 0 | 0 | 74 | 75 |
| BC6 | TX/N2/M0 | 0 | 3 | 2 | 3 | 0 | 20 | 28 |
| BC7 | T4/N3/M0 | 0 | 0 | 0 | 0 | 1 | 9 | 10 |
| BC8 | T3/N1/M0 | 0 | 8 | 0 | 0 | 6 | 6 | 20 |
| BC9 | T4/N3/M1 | 0 | 0 | 0 | 0 | 0 | 22 | 22 |
| BC10 | T4N3/M0 | 0 | 0 | 0 | 0 | 1 | 17 | 18 |
| BC11 | TX/NX/M1 | 0 | 1 | 0 | 10 | 0 | 15 | 26 |
| BC12 | T2/N1/M1 | 0 | 3 | 2 | 2 | 0 | 17 | 24 |
| BC13 | T4/N2/M0 | 1 | 5 | 0 | 18 | 27 | 33 | 83 |
| BC14 | T4N3/M0 | 1 | 4 | 3 | 1 | 0 | 26 | 34 |
| BC15 | TX/NX/M1 | 1 | 4 | 1 | 17 | 3 | 70 | 95 |
| BC16 | TX/NX/M1 | 1 | 5 | 2 | 2 | 11 | 21 | 41 |
| BC17 | TX/NX/M1 | 3 | 12 | 0 | 0 | 2 | 70 | 84 |
| BC18 | T2/N1/MX | 8 | 9 | 1 | 0 | 4 | 11 | 25 |
| BC19 | TX/NX/M1 | 11 | 9 | 0 | 0 | 0 | 21 | 30 |
| BC20 | T4/N3/M0 | 24 | 12 | 16 | 0 | 14 | 34 | 76 |
| BC21 | T1/N2/M0 | 112 | 90 | 18 | 6 | 0 | 28 | 142 |
| Average ± SD | | 7.7 ± 4.6 | 8.1 ± 19.2 | 2.1 ± 5.0 | 3.2 ± 5.4 | 27.6 ± 21.4 | 5.5 ± 11.2 | 46.6 ± 37.0 |
| Median | | 0 | 3.0 | 0 | 1 | 21 | 1.0 | 28.0 |
| PC1 | T2/N0/M0 | 0 | 2 | 0 | 0 | 0 | 5 | 7 |
| PC2 | TX/NX/M1 | 0 | 0 | 2 | 0 | 0 | 19 | 21 |
| PC3 | TX/NX/M1 | 1 | 1 | 0 | 0 | 4 | 62 | 67 |
| PC4 | T3NX/MX | 4 | 3 | 0 | 3 | 1 | 18 | 25 |
| PC5 | T2/N0/M0 | 7 | 0 | 5 | 0 | 0 | 24 | 29 |
| PC6 | T3/N1/M1 | 9 | 0 | 18 | 12 | 0 | 17 | 47 |
| PC7 | T3/N0/MX | 10 | 6 | 6 | 5 | 0 | 23 | 40 |
| PC8 | T2/N1/M0 | 10 | 8 | 0 | 0 | 2 | 18 | 28 |
| PC9 | TX/NX/M1 | 73 | 34 | 31 | 23 | 0 | 27 | 115 |

| | | | | | | | |
|---|---|---|---|---|---|---|---|
| Average ± SD | 12.7 ± 23.0 | 6.0 ± 10.9 | 6.9 ± 10.7 | 4.8 ± 7.9 | 23.7 ± 15.7 | 0.8 ± 1.4 | 42.1 ± 32.2 |
| Median | 7.0 | 2.0 | 2.0 | 0.0 | 19.0 | 0.0 | 29.0 |
| Total Average | 9.2 ± 23.8 | 7.5 ± 16.9 | 3.6 ± 7.4 | 3.7 ± 6.1 | 26.4 ± 19.6 | 4.1 ± 9.6 | 45.3 ± 35.1 |
| Total Median | 1.0 | 3.0 | 0.0 | 0.5 | 20.5 | 0.0 | 28.5 |

To compare the two assays, method comparison analyses were run using linear regression plots with correlation significance. A sample size of 30 was selected which gives the statistical power necessary to detect differences smaller than the intrinsic variability range of the Cell Search Test. When comparing regression plots between the total CK+/CD45− population isolated by CellSieve™ versus CellSearch®, it was found that the cells were not equivalent, whether EpCAM presence was included or not—Table 3.

TABLE 3
Comparison of CellSieve ™ capturing different CTC morphologies with
CellSearch ®

| | Total CK+ | | | | | |
|---|---|---|---|---|---|---|
| | cells | PDCTC | EACTC | LACTC | EMTCTC | Atypical CK+ |
| | vs. | vs. | vs. | vs. | vs. | cells vs. |
| Comparison | CellSearch ® | CellSearch ® | CellSearch ® | CellSearch ® | CellSearch ® | CellSearch ® |
| R² | 0.4427 | 0.9107 | 0.6033 | 0.1311 | 0.0108 | 0.0002 |
| p-value | 6.03 * 10⁻⁵ | 3.18 * 10⁻¹⁶ | 4.50 * 10⁻⁷ | 0.05 | 0.58 | 0.95 |

This lack of equivalency matches most previous studies regarding the comparison of CellSearch® to other techniques. However, when individual CellSieve™ subpopulations were compared with CellSearch®, it was found that the PDCTC subgroup showed significant correlation with CellSearch® (R²=0.9107, p<0.0001; FIG. 4B. Additionally, the inclusion of EACTCs with the PDCTCs gave the best equivalency, higher than the inclusion of PDCTC with EpCAM+ expression (Tables 1A and 1D). This data suggests that the PDCTC subpopulation, regardless of EpCAM presence, is most statistically correlated to CellSearch®, while the other CK+/CD45− cells are not. Furthermore, this data suggests that although the CellSearch® system relies on capturing EpCAM+ cells for isolating CTCs, correlation of this clinically relevant CTC subtype, identified using microfiltration, is primarily dependent on cytokeratin and nuclear morphologies, and not EPCAM expression (Tables 1A-1D).

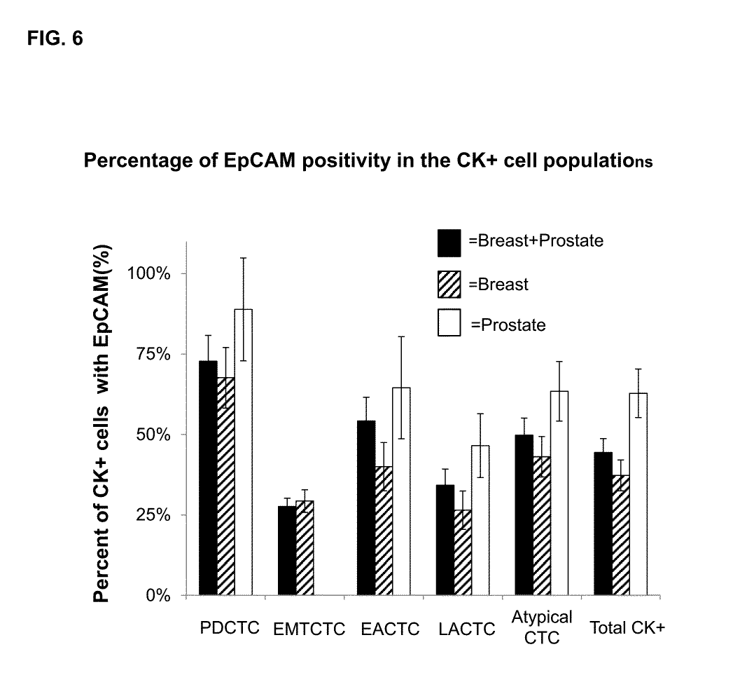

By analyzing the presence of EpCAM in the PDCTCs cells, the data appears to be in agreement with staining studies of primary biopsies which analyzed EpCAM expression. This study showed that 99% of prostate carcinomas and 74% of breast carcinomas were EpCAM positive (8). The data here shows that the percentage of EpCAM positivity in breast PDCTCs is 68% and in 90% in prostate PDCTCs (FIG. 6). Slopes and correlations of EpCAM subcategories are found in Table 1D. Error bars indicate standard error of the mean. The EpCAM data also seems to agree with the theories regarding EMT cell transition as there was less EpCAM present in cells that also have diminished CK staining, the EMT-like CTCs. However, as there in no universal definition of EMT, further analysis of the CTCs exhibiting these characteristics need to be performed when specific markers of EMT cells have been identified.

Once method correlations were established, a preliminary evaluation of the prognostic significance of the CK+ categories were performed using ≧5 CTCs/sample as a threshold for patient overall survival (OS). The criteria for clinical utility, for breast and prostate cancers, is the cut off value of 5 CTCs/sample, <5 showing longer OS than ≧5 CTCs.

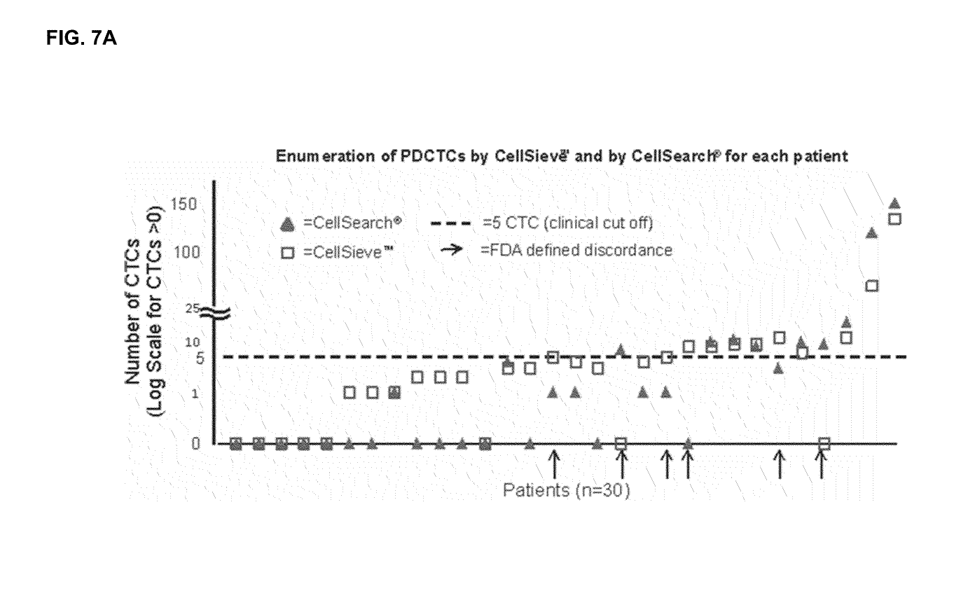

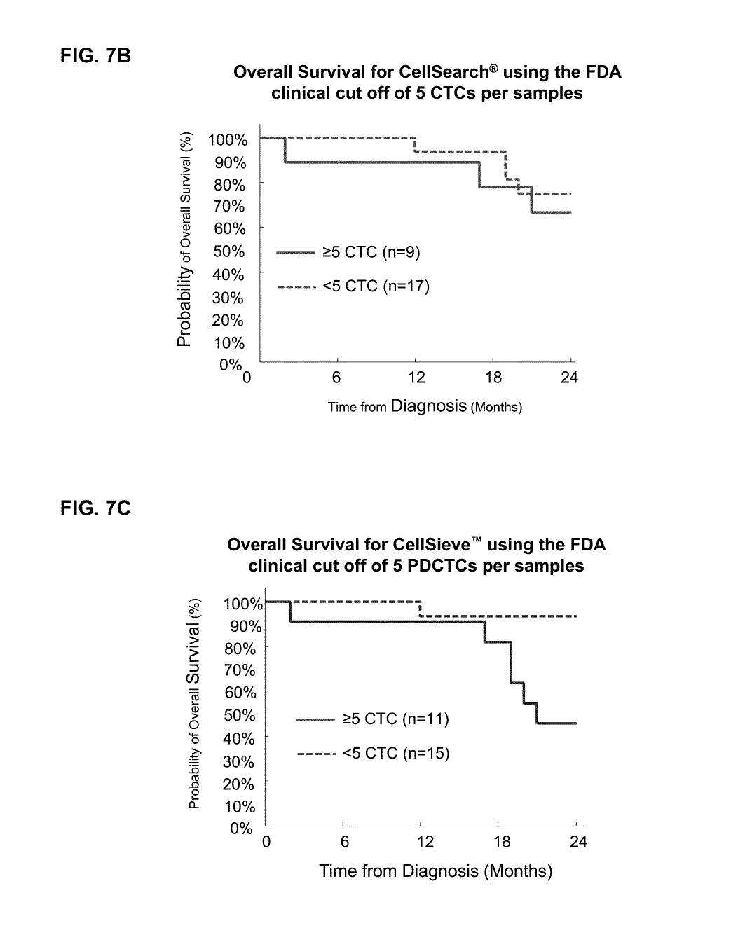

FIG. 7A shows the number of CellSieve™ PDCTCs and CellSearch® CTCs per 7.5 mL of blood. The dashed line denotes the FDA defined clinical cut-off of ≧5 CTCs for cutoff of overall survival (OS) by CellSearch. Disagreement of OS was found for six patients (n=3 breast, n=3 prostate).

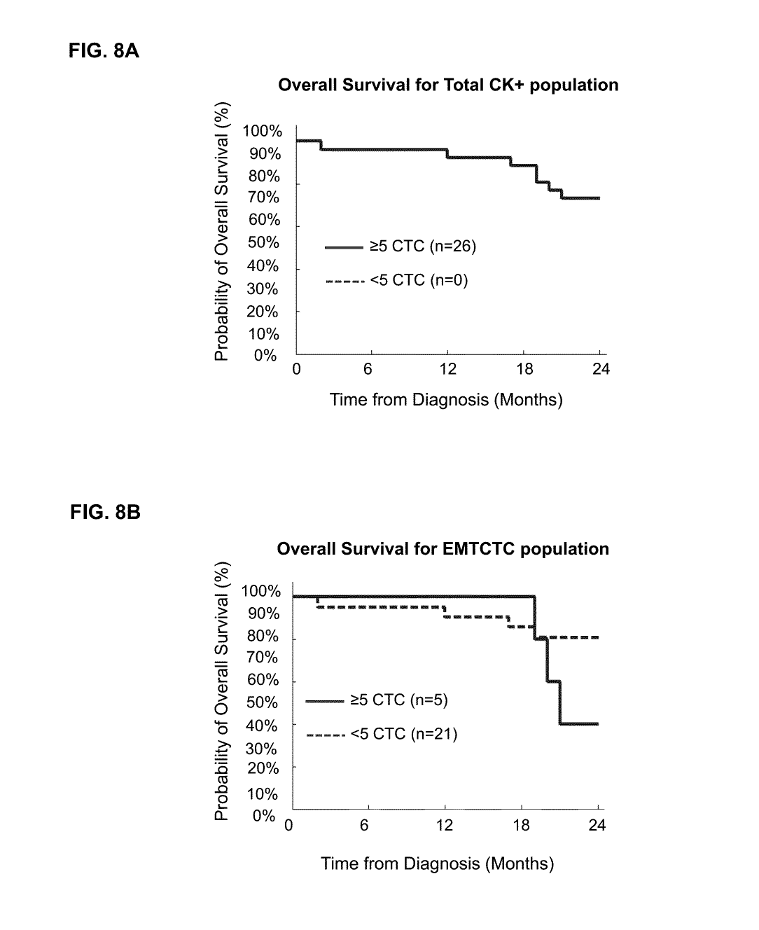

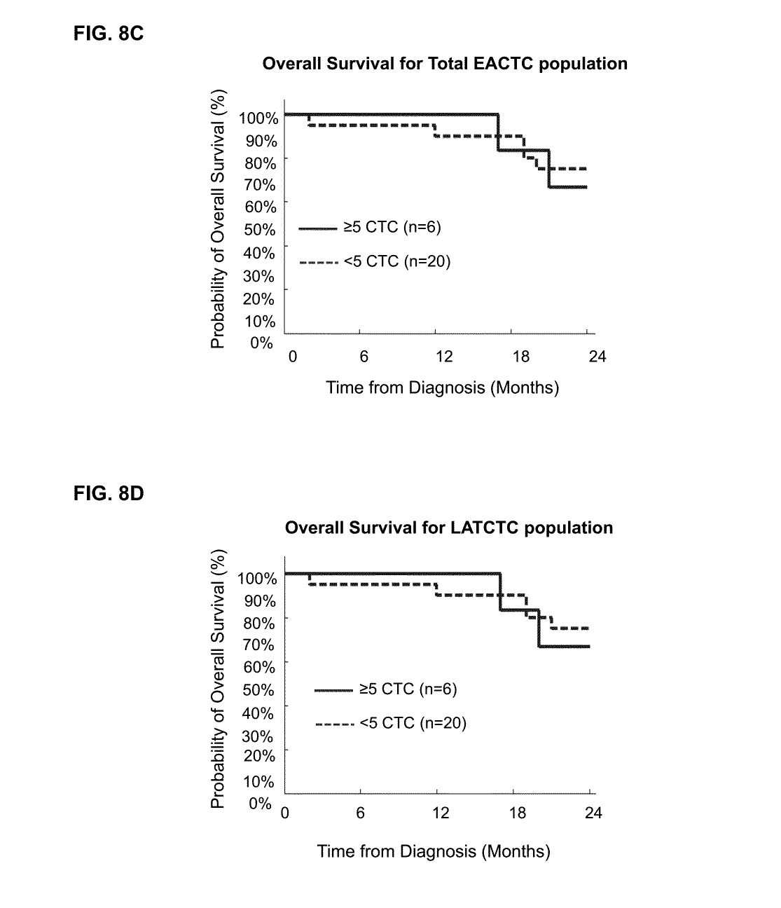

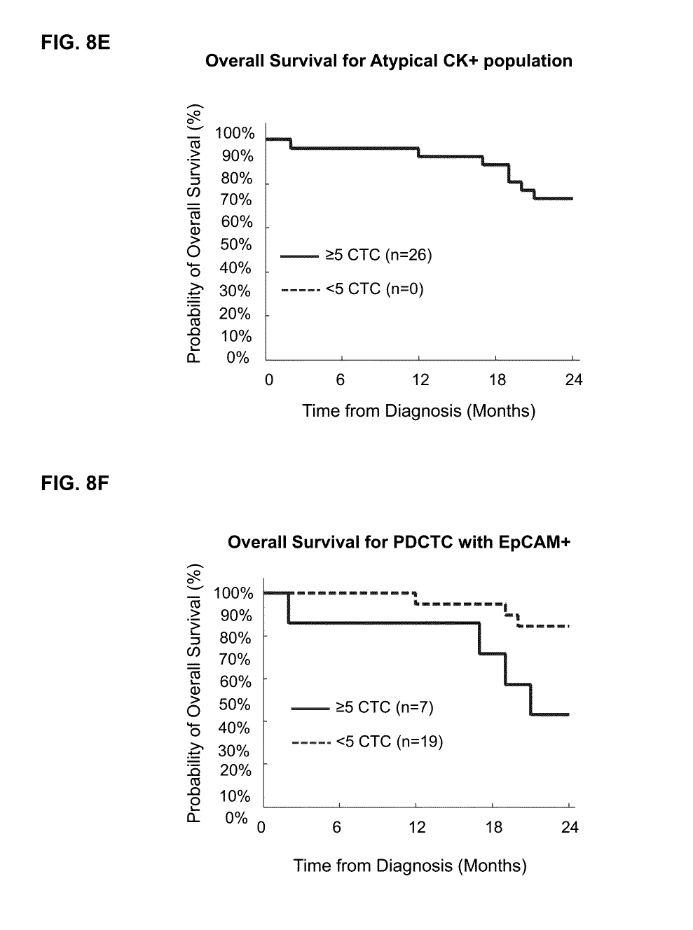

FIGS. 7B-C and FIGS. 8A-F show the survival of the 26 patients that remained on study for a 24 month period. Using the ≧5 threshold, it was found that CellSieve™ PDCTCs and CellSearch® matched in 23 of the 26 patients, and in the three instances where the methods differed, there was an observed change in the survival outcome. Additionally, both EMTCTCs and EpCAM positive PDCTCs had some lower correlations to CellSearch®, and both groups also showed some difference in overall survival for patient cohorts using the ≧5 cell criteria as shown in FIGS. 8A-F. The OS curves using PDCTC in FIG. 7B indicates that the ≧5 cell criteria indeed has strong correlation for short overall survival and is in agreement with CellSearch®. Though this data implies differences in outcome between patient cohorts, the data set is too small to draw any statically relevant conclusions. It does, however, suggest that additional larger studies may be warranted to determine if these survival trends continue to differentiate patient populations.

For many years the goal of CTC work has revolved around the concept of using blood as a “liquid biopsy” for cancer diagnosis, prognosis and treatment response. Generally, histological review of biopsies define the presence of tumor cells using morphological criteria based on organ specific histopathological grading schema describing cellular features (e.g. nuclei abnormalities, mitotic proliferation, hyperactive Golgi, etc.). However, current CTC capture techniques lack the ability to provide adequate numbers of circulating epithelial cells in a format where standard histological staining can be applied, and reviewed by a pathologist. Here it is demonstrated that multiple populations of CTCs can be identified by histopathological staining patterns of CK and DAPI using filter based isolation. These preliminary data suggest that CTCs with malignant nuclear morphologies and filamentous cytokeratin are, at least numerically, the same cells identified using CellSearch®. These findings support the hypothesis that both CellSearch® and CellSieve™ microfiltration are capable of identifying a similar number of highly specific and clinically relevant CTC subtypes.

As CTC isolation methods have become more varied and our biological understanding become greater, the defining criteria for what cells meet the designation of a CTC has become less stringent. Complicating the criteria of CTCs is the knowledge that cancer cells can undergo EMT, which has no universal definition, though generally described by the down regulation of epithelial proteins, such as EpCAM and cytokeratin. As there in no scientific consensus in the EMT definition, and not within the scope of this manuscript, an attempt to identify the EMT processes in cells was made. Instead, only an effort to describe EMT-like cells by the visual loss of filamentous structure.

When assessing new technologies one must determine the proposed usage of the capture events. If the intent is to collect product for downstream mutational analysis, this is quite different than using a new technique as a prognostic indicator of overall survival, such as CellSearch®. The primary result of many CTC capture methods is to show discordance with the clinical validity of CellSearch®, by virtue of increased CTC number. However, a fact which is largely ignored by comparative technologies is the fact that CellSearch® captures numerous cytokeratin positive particles which are known to provide prognostic value, but are excluded by the morphological identification of a trained operator. Groups typically bypass the morphological criteria, and explain this difference in CTC number between their techniques and CellSearch® through the use of additional biomarkers, e.g. EMT markers, apoptotic markers, proliferation markers. However, to date, studies focusing on these functional biological markers have lacked the ability to correlate to CellSearch® and, as such, have offered few insights into the CTC subpopulation that CellSearch® enriches for. Here, rather than focusing on the identification of the biological differences between two CTC capture technologies by using differing biomarkers, provided is the first example of matched samples, using accepted markers, which can replicate the data demonstrated using the CellSearch® system. This data suggests that size exclusion techniques coupled with characterization of specific staining morphologies might be used to identify a validated and clinically relevant CTC subpopulation for breast and prostate cancer. This exploratory study reveal an opportunity to now expand and define the clinical relevance of additional CTC subpopulations captured by non-EpCAM based techniques and better understand the CTCs CellSearch® captures.

It has been shown that the pore diameter varying from 6-8 μm did not have significant effect on the performance of the CTC isolation (Adams D L, et al. The systematic study of circulating tumor cell isolation using lithographic microfilters. RSC Adv 2014, 4:4334-4342).

What is claimed is:

1. A method for predicting overall survival of a patient having cancer, comprising enumerating pathologically-defined circulating tumor cells (PDCTCs) in a blood sample from the patient, wherein when five or more PDCTCs are present in 7.5 ml of blood, the overall survival of the patient is predicted to be lower than a patient having cancer with four or less PDCTCs present in 7.5 ml of blood, and wherein the PDCTCs are cytokeratin (CK) 8, 18, 19⁺, CD45⁻, DAPI⁺ cells, possess a malignant nucleus, and display a filamentous CK pattern.

2. The method of claim 1, wherein the cancer is breast cancer.

3. The method of claim 1, wherein the PDCTCs are enumerated by (i) isolating CTCs from the blood sample using a filter having pores of 7-8 microns in diameter and (ii) counting PDCTCs present in the isolated population of CTCs.
